(19) United States
(12) Patent Application Publication
(10) Pub. No.: US 2013/0125910 A1
(43) Pub. Date: May 23, 2013

(54) Use of Electrophoretic Microcapsules in a Cosmetic Composition

(71) Applicant: Amitabh Bansal, Hoboken, NJ (US)

(72) Inventor: Amitabh Bansal, Hoboken, NJ (US)

(73) Assignee: AVON PRODUCTS, INC., New York, NY (US)

(21) Appl. No.: 13/299,745

(22) Filed: Nov. 18, 2011

(51) Int. Cl. A45D 40/18; A45D 44/00; A61K 8/29; A61Q 19/00; A61K 8/11; A61K 8/19

(52) U.S. Cl. 132/200; 424/401; 424/646; 132/317

(57) ABSTRACT

Provided are cosmetic compositions incorporating electrophoretic colorants than can effect stable and reversible changes in the color of the cosmetic in response to an electric field. The compositions provide for stable and reversible wearer determined changes in cosmetic color for lips, skin, hair and nails.

### FIELD OF INVENTION

The present invention relates generally to color-changing cosmetics. More specifically, the invention relates to cosmetic compositions that have electrophoretic colorants incorporated into their cosmetically acceptable carrier that permit the wearer to affect a stable change in the color (hue, tone, and/or intensity) of the cosmetic by applying an electric stimulus to the cosmetic.

### BACKGROUND OF THE INVENTION

While consumers seek cosmetics that are personalized for them, a need persists for cosmetics whose color (hue, tone, shade, intensity, etc.) may be adjusted for their individual needs once applied.

Color changing cosmetics have existed for a number of years. For example, cosmetics incorporating weak acid pigments or thermochromatic pigments have been sold as “mood” or personalized cosmetics for a number of years. These cosmetics change color in response to the individual wearer's pH, i.e. the weak acid pigment changes color as it is neutralized, or temperature, respectively. Examples of these types of cosmetics are sold as L'Paige™ Cosmetics Lipsticks, Mood Matcher™ Lipsticks from Fran Wilson, Mood Lips Color Changers, Cherry Culture's Amuse Fruit Lipsticks, Aloe Mood Lips and DuWop™ Private red lipsticks. Photochromatic pigments have also been incorporated into some cosmetics, namely fashion/press-on nails, to effect a color change upon exposure to UV rays. Additionally, luster pigments have been incorporated into cosmetics to effect a color shift in the cosmetic when it is viewed from different angles such as the borosilicate pigments incorporated into Englehard Corporation (Iselin, N.J.) Reflecks™ MultiDimension Pigments. Further, cosmetics may irreversibly change colors in response to stimulus such as friction such as Krylon's eye shadow.

However, none of these existing color changing cosmetics provide the wearer with complete control over the color of their cosmetics. For example, the weak acid and thermochromatic pigments color shift cannot be known until it is applied to the wearer's skin. Further, these color shifts may not be stable, i.e. photochromatic pigments revert to their initial color in the absence of UV rays and luster pigments would need to be viewed at a constant angle to retain the desired color. Lastly, many of these pigments are irreversible so that if the wearer does not like the color change effected by the pigment they would need to modify the pigment with additional cosmetics or remove the cosmetic altogether and reapply. Thus, a need remains for a color changing cosmetic that allows for a stable but reversible color change to suit the wearer's needs.

In electric media, electrophoretic particles, charged particles dispersed within a carrier that move under the influence of an electric field, have been used to effect color changes within various electronic displays. Numerous patents directed to the technology have been filed by Ink Holdings and Massachusetts Institute of Technology, a representative sampling of which includes, U.S. Pat. Nos. 5,930,026; 5,961,804; 6,017,584; 6,067,185; 6,118,426; 6,120,588; 6,120,839; 6,124,851; 6,130,773; 6,130,774; 6,172,798; 6,177,921; 6,232,950; 6,249,271; 6,252,564; 6,262,706; 6,262,833; 6,300,932; 6,312,304; 6,312,971; 6,323,989; 6,327,072; 6,376,828; 6,377,387; 6,392,785; 6,392,785; 6,413,790; 6,422,687; 6,445,374; 6,445,489; 6,459,418; 6,473,072; 6,480,182; 6,498,114; 6,504,524; 6,506,438; 6,512,354; 6,515,649; 6,518,949; 6,521,489; 6,531,997; 6,535,197; 6,538,801; 6,545,291; 6,580,545; 6,639,578; 6,652,075; 6,657,772; 6,664,944; 6,680,725; 6,683,333; 6,704,133; 6,710,540; 6,721,083; 6,724,519; 6,727,881; 6,738,050; 6,750,473; 6,753,999; 6,816,147; 6,819,471; 6,822,782; 6,825,068; 6,825,829; 6,825,970; 6,831,769; 6,839,158; 6,842,167; 6,842,279; 6,842,657; 604,875; 6,865,010; 6,866,760; 6,870,661; 6,900,851; 6,922,276; 6,950,200; 6,958,848; 6,967,640; 6,982,178; 6,987,603; 6,995,550; 7,002,728; 7,012,600; 7,012,735; 7,023,430; 7,030,412; 7,030,854; 7,034,783; 7,038,655; 7,061,663; 7,071,913; 7,075,502; 7,075,703; 7,079,305; 7,106,296; 7,109,968; 7,110,163; 7,110,164; 7,116,318; 7,116,466; 7,119,759; and 7,119,772; and U.S. Patent Applications Publication Nos, 2002/0060321; 2002/0090980; 2002/0180687; 2003/0011560; 2003/0102858; 2003/0151702; 2003/0222315; 2004/0014265; 2004/0075634; 2004/0094422; 2004/0105036; 2004/0112750; 2004/0119681; 2004/0136048; 2004/0155857; 2004/0180476; 2004/0190114; 2004/0196215; 2004/0226820; 2004/0239614; 2004/0257635; 2004/0263947; 2005/0000813; 2005/0007336; 2005/0012980; 2005/0017944; 2005/0018273; 2005/0024353; 2005/0062714; 2005/0067656; 2005/0078099; 2005/0099672; 2005/0122284; 2005/0122306; 2005/0122563; 2005/0122565; 2005/0134554; 2005/0146774; 2005/0151709; 2005/0152018; 2005/0152022; 2005/0156340; 2005/0168799; 2005/0179642; 2005/0190137; 2005/0212747; 2005/0213191; 2005/0219184; 2005/0253777; 2005/0270261; 2005/0280626; 2006/0007527; 2006/0024437; 2006/0038772; 2006/0139308; 2006/0139310; 2006/0139311; 2006/0176267; 2006/0181492; 2006/0181504; 2006/0194619; 2006/0197736; 2006/0197737; 2006/0197738; 2006/0198014; 2006/0202949; and 2006/0209388; and International Applications Publication Nos. WO 00/38000; WO 00/36560; WO 00/67110; and WO 01/07961; and European Patents Nos. 1,099,207 B1; and 1,145,072 B1. Using these displays binary color changes may be effected through the use of single particle electrophoretic displays where the charged particle is of a first color and the carrier medium is dyed to a second color. If the electric field repels the particle the display will exhibit the second color, and if the electric field attracts the particles the display will exhibit the first color. A multiple particle display may achieve a greater range of color changes through the use of two or more electrophoretic particles. For example, if the carrier contained oppositely charged red and black particles the display would exhibit brown in a neutral state but either red or black under the influence of an electric field. These electrophoretic displays are known for their brightness and contrast, wide viewing angles, bistability, the ability to maintain the color change for an extended period of time after the electric field is removed, and low power consumption. However, to date electrophoretic particles have not been used to effect a stable and reversible color change in applied cosmetics.

It is therefore an object of the invention to provide improved cosmetic and personal care products that allow the wearer to make stable and reversible changes within the cosmetics they apply.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

In a first embodiment of the invention, a stable and reversible color-changeable cosmetic composition for application to a human integument having a first color when applied has at least one electrophoretic colorant having a color, a charge, and a zeta potential within a suspension medium, wherein when a first electric field source having a charge is placed in proximity to the color-changeable cosmetic the electrophoretic colorant moves relative to the first electric field source within a desired time to effect a change in the color-changeable cosmetic to a second color which may be further modified upon application of a second electric field source.

In a further embodiment, the suspension medium may be a suitable cosmetic vehicle and may have an initial viscosity of less than about 100,000 centipoise. Further, the suspension may be opaque in color and may form the first color for the color-changeable cosmetic, wherein the electrophoretic colorant would provide the color for the second color of the cosmetic composition.

In a further embodiment the color-changeable cosmetic composition may have more than one electrophoretic colorant wherein each electrophoretic colorant has a different color, in particular, the cosmetic composition may be further comprised of two electrophoretic colorants, a first electophoretic colorant having a first electrophoretic colorant color and a first electrophoretic charge and a second electrophoretic colorant having a second electrophoretic colorant color and a second electrophoretic colorant charge. In another embodiment, the first electrophoretic colorant charge is different than the second electrophoretic colorant charge.

In yet another embodiment, the electrophoretic colorants have zeta potential greater than about 2 mV and, preferably greater than about 10 mV. Further, where the electrophoretic colorants have the same charge the electrophoretic colorants have non-overlapping zeta potentials, and preferably the zeta potentials are separate by at least 2 mV.

The first electric field source is the human integument, and preferably the skin

Another embodiment relates to the use of a microcapsule to encapsulate the at least one electrophoretic colorant and the suspending medium. The microcapsule is preferably cubical, cylindrical, or spherical in shape, and has a diameter of less than about 200 μm, preferably less than about 100 μm, and most preferably less than about 50 μm.

Another embodiment of the invention is a method utilizing the color changeable cosmetic of the current invention on a person's integument. In a further embodiment, the method may be used as a way to identify damaged or injured skin comprised of applying the above-noted color-changeable cosmetic to a portion of skin of an individual in need thereof, wherein the color-changeable cosmetic adopts the second color when applied over damaged or injured skin. The damaged or injured skin is selected from the group consisting of chronologically aged skin, photo-aged skin, hormonally aged, and/or actinic aged skin, atrophied skin, areas with impaired microcirculation, cracked skin or areas where the skin barrier has been impaired, bruised, fatigued and/or stressed skin; and environmentally stressed skin. The damaged or injured skin may further be skin suffering from or at risk of developing an affliction or a malady, and those afflictions may include skin cancer, skin lesions, acne, psoriasis, or warts.

A further embodiment of the invention relates to a cosmetic kit having a color changeable cosmetic composition of the current invention and a triboelectric field source applicator having a case having a mating surface therein; a wand having a head surface thereon; wherein the mating surface and head surface are made of materials possessing different charges and when the head surface is rubbed against the mating surface a charge is generated. In a preferred embodiment the head surface is made of Teflon.

### BRIEF DESCRIPTION OF THE DRAWINGS

FIG. 1 illustrates a colorant capsule of the color changeable cosmetic of the current invention.

FIG. 2 illustrates a colorant capsule of the color changeable cosmetic of the current invention containing a single electrophoretic colorant and a colored internal medium and the resulting color change when an electric field is applied.

FIG. 3 illustrates a colorant capsule of the color changeable cosmetic of the current invention containing two electrophoretic colorants having opposite charges and the resulting color change when an electric field is applied.

FIG. 4 illustrates the shade changes that can occur within colorant capsules possessing several neutral colorant particles in the presence of two electrophoretic colorants (black and white) having opposite charges (positive and negative) when exposed to electric fields of varying charge and intensity.

FIG. 5 illustrates a triboelectric wand suitable for applying an electric field to the cosmetic compositions of the current invention having a configuration similar to that of a compact.

FIG. 6 illustrates a closed configuration for a triboelectric wand having a configuration similar to a mascara tube.

FIGS. 7a and 7b illustrate the use of the color-changeable cosmetics of the current invention to identify damaged areas of the skin.

### DETAILED DESCRIPTION

All terms used herein are intended to have their ordinary meaning unless otherwise provided.

As used herein, the term “consisting essentially of” is intended to limit the invention to the specified materials or steps and those that do not materially affect the basic and novel characteristics of the claimed invention, as understood from a reading of this specification. All percentages are by weight based on the total weight of the composition, unless otherwise indicated.

The present invention provides for color changeable cosmetic compositions incorporating electrophoretic colorants in cosmetically acceptable carriers which can stably change colors in response to an electric field: the electric charge of skin (naturally or artificially charged) or an external field (such as a charged wand). Cosmetic compositions of the current invention provide for an on demand color shift (color, shade, or intensity) in areas of the skin, hair, nails, etc. desired by the wearer. Further, the cosmetics may be sensitive to changes in the skin's natural electric field thereby providing a cosmetic capable of providing a natural pixilated appearance or a means of revealing damaged skin and/or neutralizing the damaged skin's appearance.

The color changeable cosmetic composition of the current invention generally encompasses electrophoretic colorants suspended within a suspension media. In its simplest form, the cosmetic compositions of the current invention encompass electrophoretic colorants, i.e. colorants having a charge and exhibiting electrophoretic mobility (zeta potential) sufficient to effect a color change within a desired time period, suspended in a suspension medium, typically a cosmetically acceptable vehicle. In another embodiment, the electrophoretic colorants of the current invention are suspended within a suspension liquid and encapsulated to protect against the colorants settling within the cosmetic and assure their even distribution within the cosmetic. For clarity's sake, when the electrophoretic colorants are not encapsulated the suspension medium and cosmetic vehicle are synonymous; whereas when the electrophoretic colorants are encapsulated the suspension medium refers to the medium within the capsule and may be different than the cosmetic vehicle.

The wearer can change the color of the cosmetic by applying an electric field to the color-changeable cosmetic such that the electrophoretic colorants move relative to the electric field. In particular, charges are attracted to electric fields of the opposite charge and thus a negative field will attract positive electrophoretic colorants and vice versa. Further, charges are repelled by electric fields of the same charge, thus a positive field will repel a positively charged electrophoretic colorant. Thus a charged electrophoretic colorant's movement relative to the electric field will be towards the source of the field or away from the source of the electric field in the presence of an attractive electric field or repellant electric field, respectively. Using these principles, several different color combinations, as illustrated below, may be achieved by pushing or pulling the electrophoretic colorants towards or away from the display surface, i.e. the outward facing surface of the cosmetic visible to consumers.

The cosmetic compositions of the current invention are stable, i.e. the color state will remain stable for a period of time, preferably over the useful life of the cosmetic composition once the electric field is removed or until an electric stimulus is applied again. The definition of stable depends upon the application for the cosmetic. For example, for cosmetic applications such as lipstick, rouge, foundation etc. the useful life would be over the period of hours the make-up would be worn, typically about 1-24 hours, preferably about 4-20 hours, and more preferably about 8-16 hours, whereas for uses such as nail polish the color state may need to remain stable over a number of days or weeks.

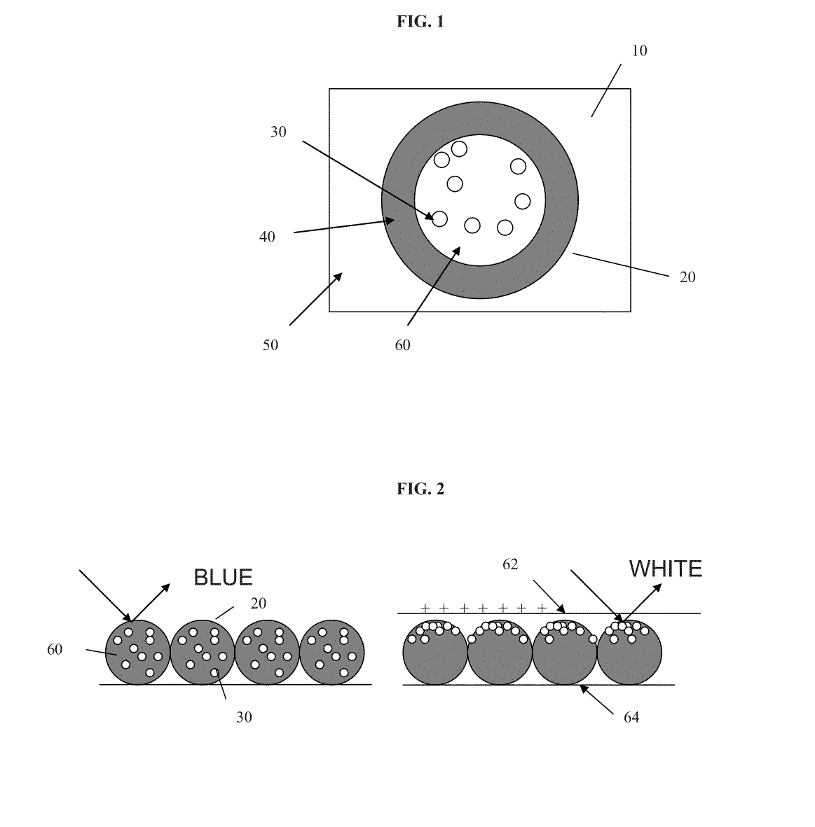

FIG. 1 generally depicts the structure of the encapsulated embodiment of cosmetics of the current invention. Specifically, the cosmetic 10 of the current invention uses a colorant microcapsule 20 dispersed within an external medium 50. Within the microcapsule 20 an electrophoretic colorant 30 is suspended within a suspension medium 60. The electrophoretic colorant 30 may include one or more colorants of (1) different colors, hues, and/or shades, (2) different charges, and/or (3) different electrophoretic mobility (zeta potential) to achieve different colors as disclosed in the following embodiments. For ease going forward the electrophoretic colorants will be referred to by color (charge).

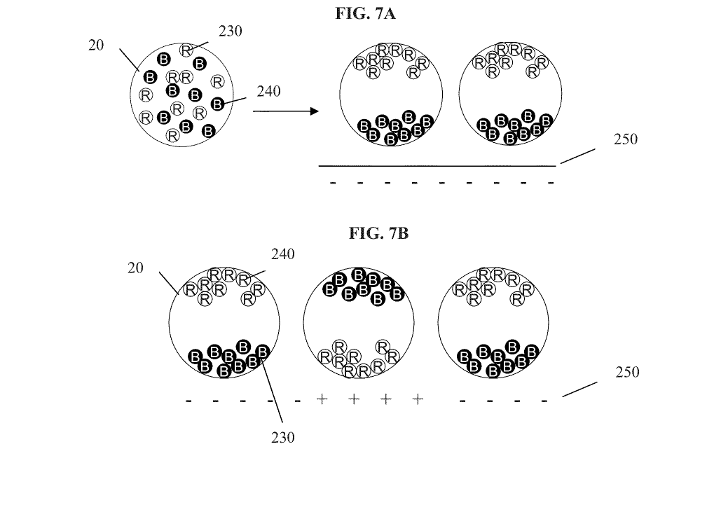

An embodiment incorporating a single electrophoretic colorant is depicted in FIG. 2. Within this embodiment, the internal/suspension medium 60 of the microcapsule 20 is an opaque color, such as blue. A single electrophoretic colorant 30 of a color different than the internal/suspension medium 60, such as white, is suspended within the internal/suspension medium 60. In the absence of an electric stimulus, the microcapsule 20 will adopt the color of the opaque internal/suspension medium 60, i.e. blue within the current example. When an attracting electric field is applied to a portion of the microcapsule the color displayed on the display surface changes in response. For example, the display surface 62, of the microcapsule 20 will adopt the color of the electrophoretic colorant 30, i.e. white in this example. Alternatively, the contact surface 64, i.e., the surface typically opposite the display surface which is proximal to the portion of the cosmetic in contact with the wearer or bottom of the container or display surface in which the cosmetic is contained, can be subjected to a repelling electric field, forcing the electrophoretic colorant to the opposing face, i.e. the display surface, and giving the microcapsule the color of the electrophoretic colorant, as shown in FIG. 7b discussed in further detail below. This embodiment allows for a binary color change.

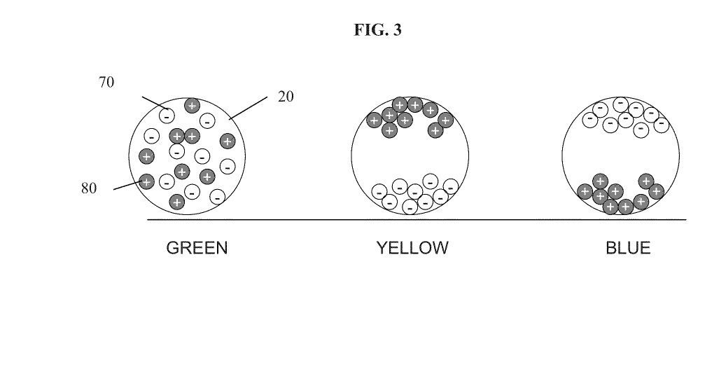

In a further embodiment of the current invention, the color-changeable cosmetic contains two or more electrophoretic colorants to achieve a wider color palette for the cosmetic. FIG. 3 illustrates a color-changeable cosmetic in which two electrophoretic colorants are present. A first electrophoretic colorant having a first color, a first charge, and a first zeta potential and a second electrophoretic colorant having a second color, a second charge, and a second zeta potential. Within FIG. 3, the first is a yellow (+) electrophoretic colorant 80, and the second is a blue (−) electrophoretic colorant 70. As shown in FIG. 3, the neutral state of the colorant microcapsule 20 depicts a green color the secondary color achieved from the combination of the primary colors blue and yellow. Upon application of a negative electric field, an attractive field for the yellow (+) electrophoretic colorant 80 and a repellant field for the blue (−) electrophoretic colorant 70, the colorant microcapsule 20 will change yellow in color as the yellow (+) electrophoretic colorants 80 migrate to the top of the colorant microcapsule and the blue (−) electrophoretic colorant 70 settles to the bottom of the colorant microcapsule. Conversely, the colorant microcapsule 20 will adopt a blue color if a positive electric field is applied as the blue (−) electrophoretic colorants 70 are attracted to the top of the colorant microcapsule and the yellow (+) electrophoretic colorants 80 settle to the bottom of the microcapsule 20. In this manner, the number, proportion, and color of electrophoretic colorants may be varied so as to provide a full pallet of colors consistent with the RGB or CMYK color charts.

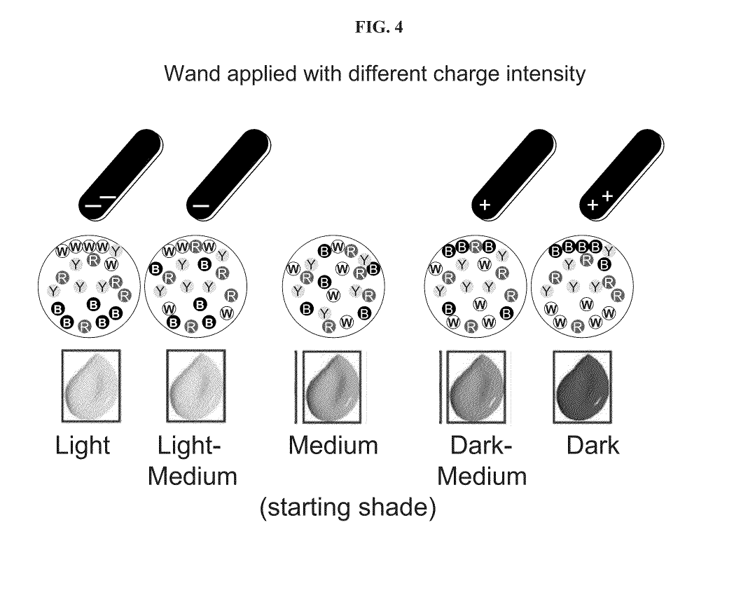

The cosmetic of the current invention may also be used to achieve several different shades of a color as well. FIG. 4 illustrates this particular embodiment. In particular, the capsules of FIG. 4 encapsulate two different electrophoretic colorants: a black (−) electrophoretic colorant 100, and a white (+) electrophoretic colorant 110; and two different non-electrophoretic colorants: a red (neutral) electrophoretic colorant and a yellow (neutral) electrophoretic colorant 120. Depending upon the level of electric field, different amounts of particles will migrate to the surface or settle at the bottom. Consequently, a shade palate can be developed. This is shown within FIG. 4, the cosmetic composition will initially exhibit the secondary color achieved through the combination of the red and yellow colorants and a shade Medium as shown in FIG. 4. If a negative electric field, an attracting field for the white (+) electrophoretic colorant 110 and repelling field for the black (−) electrophoretic colorant 100, is applied the secondary color will lighten generating a Light-Medium shade. Further, if the negative electric field is stronger or applied to the cosmetic for a longer period of time the secondary color will be lightened further generating a Light shade. Conversely, if a positive electric field is applied to the cosmetic, the secondary color will darken generating a Dark-Medium shade, and if a stronger electric field is applied or the field is applied for a longer period of time the secondary color will darken further generating a Dark shade.

Further, colors may be achieved by varying the zeta potentials, electrophoretic of the constituent electrophoretic colorants. For example, in situations where the internal/suspension medium is opaque the electrophoretic colorants may exhibit the same charge (+/−) but have varying zeta potentials. Thus, when the attracting or repelling electric fields are applied several different colors may be achieved as the electrophoretic colorants having the highest zeta potentials will migrate first, the next highest zeta potential will migrate next, and so on with the least zeta potential migrating last. For example, if the colorant capsule had a white internal/suspension medium and contained a red (+) electrophoretic colorant having a zeta potential of 40 MV, a blue (+) electrophoretic colorant having a zeta potential of about 30 mV, and a black (+) electrophoretic colorant having a zeta potential of about 20 mV. In its neutral state, the color capsule would exhibit a white/gray color and upon application of an attracting (−) electric field the colorant capsule will exhibit a red color as the red colorant migrates to the top of the capsule first and after a further period purple as the blue colorant migrates to the top, and after a further period of time a darker shade of purple as the black colorant migrates to the top. Once all of the electrophoretic colorants have been drawn to the top, further colors may be achieved by applying a repellant (+) electric field as the colorant capsule will first adopt a deep shade of blue, and then a black color before reverting to the original white/grey color.

Electrophoretic Cosmetic Compositions

As noted above, the simplest cosmetic of the current invention is comprised of at least one electrophoretic colorant suspended in a suspension medium, which in this case is the cosmetic vehicle. In preferred embodiments, at least one colorant microcapsule, a capsule surrounding at least one electrophoretic colorant suspended within a suspension medium, is used. These colorant capsules are suspended within a suitable cosmetic vehicle. In alternative embodiments, one or more electrophoretic colorants or colorant microcapsules having different colors may be used within the same cosmetic compositions to achieve various colors, color effects, or optical effects, i.e., a cosmetic composition containing two microcapsules: one with blue suspension medium and white electrophoretic colorants and a second with red suspension medium with a black electrophoretic colorant to achieve various shades of blue, red, purple, and grey. Materials and means for manufacturing such colorant capsules are generally disclosed within U.S. Pat. Nos. 6,727,881 and 7,002,728, hereby incorporated by reference in their entirety. Further materials and means particularly relevant to the field of use of the current invention, i.e. cosmetics, skin, nail, and/or hair products, etc., are disclosed below.

Electrophoretic Colorant

For purposes of this invention, various types of colorants may be used in the current invention provided that they are charged or are modified to adopt a charge and have sufficient electrophoretic mobility (zeta potential to effect a color change within a desired time period. As noted above, in certain embodiments the electrophoretic colorants may be used in conjunction with non-electrophoretic colorants, i.e. colorants lacking a charge or sufficient electrophoretic mobility to effect a color change within the desired time, to achieve various color combinations or shades of colors.

As used herein, the term “colorant” includes any material added to impart a hue or optical effect to the composition, and includes without limitation pigments, pearls, lakes, dyes, glitters, polymers, and/or combinations thereof. Electrophoretic colorants, as noted above, are those that have a charge and sufficient zeta potential to effect the desired changes within the required time period. Non-electrophoretic colorants known in the art may be modified to electrophoretic colorants by adopting a charge or enhancing their zeta potential using methods known in the art. Preferably, the colorants are cosmetically acceptable. Suitable cosmetically acceptable colorants are well known in the art and are disclosed in the C.T.F.A. Cosmetic Ingredient Handbook, First Edition, 1988, the contents or which are hereby incorporated reference.

Suitable colorants whether non-electrophoretic or electrophoretic are recited below.

Pigments—Exemplary inorganic pigments include, but are not limited to, metal oxides and metal hydroxides such as iron oxides (α-Fe₂O₃, β-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides and zinc oxides and composite oxides and composite hydroxides such as iron titanate, cobalt titanate, cobalt aluminate, ultramarine blue (i.e., sodium aluminum silicate containing sulfur), Prussian blue, manganese violet, bismuth oxychloride. Further, luminescent pigments such as zinc sulfide may be incorporated as well.
Pearls, effect pigments and Glitters—include talc, mica, sericite, titanated mica, iron oxide titanated mica, bismuth oxychloride, and the like. Further, one or more chroma-methicone colorants may be used, e.g., chroma-lite yellow-methocone, chroma-lite red-methicone, and chroma-lite black-methicone. Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference. The compositions may also include glittering agents
Dyes—FD&C dyes, D&C dyes, including D&C Red, Nos. 2, 5, 6, 7, 10, ii, 12, 13, 30 and 34, D&C Yellow No. 5, Blue No. 1, and Violet No. 2. Florescent dyes such as D&C Orange Nos. 5, 10, and 11 as well as D&C Red Nos. 21, 22, 27 and 28 may be used as well.

Lakes—Laked pigments, particles that have a dye precipitated on them or which are stained such as metal salts of readily soluble anionic dyes, may also be used as electrophoretic particles. These are dyes of azo, triphenylmethane or anthraquinone structure containing one or more sulphonic or carboxylic acid groupings. They are usually precipitated by a calcium, barium, strontium, or aluminium salt onto a substrate. Typical examples are peacock blue lake (CI Pigment Blue 24) and Persian orange (lake of CI Acid Orange 7), Black M Toner (GAF) (a mixture of carbon black and black dye precipitated on a lake).

In some embodiments, an alkyl silane surface-treated colorant comprising an alumina substrate (e.g., platelet shaped) and a pigment, dye, or lake bonded to the alumina substrate by an alkyl silane surface treatment. Typically, the alkyl silane will be octylsilane and may be formed by treatment with triethoxy caprylyisilane. Non-limiting examples of such colorants include, but are not limited to, the COVALUMINE™ line by SENSIENT™ Cosmetic Techologies LCW. The colorants may be surface modified, for example with triethoxy caprylyisilane, to adjust one or more characteristics of the colorant, such as dispersibility in the vehicle.

In a further embodiment of the current invention, the colorant may be a combination of pigments and polymers. The pigments and polymers may be randomly located within the colorant or aggregated within the colorant. Additionally, the pigment and polymer may be present in a core-shell configuration in which the pigment/dye, etc. is surrounded, completely or partially by the polymer. The combination of the polymer and pigment may serve to scatter light, absorb light, or both. Further, the polymers may impart a charge to the pigment and thereby render it electrophoretic for purposes of the current invention.

Useful polymers for the particles include, but are not limited to: polystyrene, polyethylene, polypropylene, phenolic resins, E. I. du Pont de Nemours and Company Elvax resins (ethylene-vinyl acetate copolymers), polyesters, polyacrylates, polymethacrylates, ethylene acrylic acid or methacrylic acid copolymers (Nucrel Resins—E. I. du Pont de Nemours and Company, Primacor Resins—Dow Chemical), acrylic copolymers and terpolymers (Elvacite Resins, E. I. du Pont de Nemours and Company) and PMMA. Useful materials for homopolymer/pigment phase separation in high shear melt include, but are not limited to, polyethylene, polypropylene, polymethylmethacrylate, polyisobutylmethacrylate, polystyrene, polybutadiene, polyisoprene, polyisobutylene, polylauryl methacrylate, polystearyl methacrylate, polyisobornyl methacrylate, methacrylate, polyethyl methacrylate, polymethyl acrylate, polyethyl acrylate, polyacrylonitrile, and copolymers of two or more of these materials. Some useful pigment/polymer complexes that are commercially available include, but are not limited to, Process Magenta PM 1776 (Magruder Color Company, Inc., Elizabeth, N.J.), Methyl Violet PMA VM6223 (Magruder Color Company, Inc., Elizabeth, N.J.), and Naphthol FGR RF6257 (Magruder Color Company, Inc., Elizabeth, N.J.).

The pigment-polymer composite may be formed by a physical process, (e.g., ball milling, attrition, jet milling), a chemical process (e.g., dispersion polymerization, mini- or micro-emulsion polymerization, suspension polymerization precipitation, phase separation, solvent evaporation, in situ polymerization, seeded emulsion polymerization, or any process which falls under the general category of microencapsulation may be used), or any other process known in the art of particle production.

Typical considerations for the electrophoretic colorant of the current invention are its size, optical properties, electrical properties, and surface chemistry.

The size of the colorant of the current invention should be at least about 10 times less, more preferably about 50 times less, and most preferably about 100 times less than the size of the shell. Alternatively, the colorant should be about 100 μm to about 1 nm in size more preferably about 50 μm to 250 nm, and most preferably about 10 μm to about 400 nm. Further it is possible that the colorant be a nanoparticle having a diameter substantially less than a wave length of light (less than 400 nm) provided that when the nanoparticle colorant is drawn to the electric stimulus it agglomerates forming a visible optical state, i.e. a color. See U.S. Pat. Nos. 6,538,801, 6,323,989, and 6,721,083, hereby incorporated by reference in their entirety. Furthermore, the particles need not be uniformly shaped and may be irregularly shaped in order to modify the optical properties of the particles.

The electrical properties of the colorants can be measured using Zeta Potential (ζ potential), which is a measure of the potential difference between the dispersion medium and the stationary layer of fluid attached to the dispersed particle. Higher Zeta Potential values indicate a stable colloid that is less liable to agglomerate, and also indicates a higher electrophoretic mobility of the particle. Thus, the zeta potential for colorants of the current invention must be high enough that the colorants will not agglomerate within the internal medium, and will traverse the capsule quickly enough that the desired change in color of the cosmetic will occur within a reasonable time period, less than about 10 seconds; preferably about 0.1 to 5 seconds, and most preferably 0.1 to 1 seconds. For the colorants used within the current invention, the absolute Zeta Potential should be greater than 2 in V, more preferably greater than about 10 mV, and most preferably greater than about 30 mV. Furthermore, in embodiments of the current invention in which greater than two electrophoretic colorants are present or where all electrophoretic colorants exhibit the same charge, it is preferred that the colorants have non-overlapping zeta potentials. Preferably, the colorants' zeta potentials are separated by about 2 mV, more (preferably about 5 mV. The differences in electrophoretic mobility between the colorants may be a basis for obtaining additional color outputs.

Although the electrophoretic colorants may be used uncoated where they natively possess the desired characteristics, the electrophoretic colorants may be coated with various agents to provide the particle with or enhance the colorants characteristics. For example, the surface of the colorant may also be chemically modified to aid dispersion, to improve surface charge, and to improve the stability of the dispersion, for example. Surface modifiers include organic siloxanes, organohalogen silanes and other functional silane coupling agents (Dow Corning® Z-6070, Z-6124, and 3 additive, Midland, Miss.); organic titanates and zirconates (Tyzor® TOT, TBT, and TE Series, E. I. du Pont de Nemours and Company, Wilmington, Del.); hydrophobing agents, such as long chain (C12 to C50) alkyl and alkyl benzene sulphonic acids, fatty amines or diamines and their salts or quaternary derivatives; a quartenary silane such as 3-(n-styrylmethyl-2-aminoethylamino)propyltrimethoxysilane hydrochloride (SIS6994.0), N-trimethoxysilyl propyl-n,n,n-tri-methylammonium chloride (SIT8415.0) and actadecyldimethyl(3-trimethoxy silyl-propyl)ammonium chloride (SIO6620.0) all from (iciest); a succinimde (such as OLOA 1200 from Chevron); a calcium diisopropylsalicillate; sodium sulfosuccinate (such as Aerosol OT, AOT from American Cyanamid); ethoxylates (such as Triton X-100); and amphipathic polymers which can be covalently bonded to the particle surface.

Suspension Medium

The electrophoretic colorants are then dispersed in a suspension medium using high shear such as milling, sonication or three roll milling.

The suspension medium consists of a viscous liquid or wax that, at the desired temperature, can form a stable suspension for an extended period of time. These can be chosen from paraffinic materials including but not limited to alkanes, alkenes, oils, waxes, etc. Non-limiting examples of paraffinic materials include isoparaffin, microcrystalline wax, heavy mineral oil, light mineral oil, ozokerite, petrolatum, paraffin, and polyethylene. Alternatively, the suspension medium could be aqueous, silicone or an emulsion of the types typically used in cosmetic vehicles as disclosed below. In a further embodiment wherein the suspension medium or cosmetic vehicle are emulsions the electrophoretic colorants may be present within one or more phases of the emulsion, i.e., in an oil-in-water emulsion the electrophoretic colorant may be present within the oil phase, the water phase, or both. Polyisobutene is the preferred suspension medium.

The choice of suspending medium may be based on concerns of chemical inertness, density matching to the electrophoretic colorant, charge of the suspension medium, or chemical compatibility with both the electrophoretic colorant and bounding capsule.

In particular, the suspension medium should be selected so as to prevent the settling of the electrophoretic colorants and to maintain the stability of the resulting color changes. This may be accomplished by having a sufficiently viscous internal medium, typically with a viscosity of less than 100,000 cps, preferably from about 500 to about 50,000 cps, and especially from about 1,000 to 10,000 cps. In a further embodiment, the internal medium should have a specific density that is substantially similar to that of the electrophoretic colorants to prevent the colorants from settling. Additionally, additional materials that are responsive to the electric field may also be incorporated into the internal/suspension medium to promote the stability of the color change. For example, liquid crystals may be incorporated into the internal medium. Under the influence of the electric field the liquid crystals will organize themselves into channels that will permit the migration of the electrophoretic colorants in response to the electric field. Once the electric field is removed the liquid crystals become disorganized preventing the further movement of the electrophoretic colorants.

Additionally, the suspension medium should be chosen so that it does not interfere with the electrophoretic colorants, i.e., the suspension should not be a solvent for the particular pigments or pigment/polymers, the suspension medium should not possess a charge that interferes with the charge on the electrophoretic colorants, etc.

The suspending medium may comprise a single fluid. The medium will, however, often be a blend of more than one medium in order to tune its chemical and physical properties. Furthermore, the medium may contain surface modifiers to modify the surface energy or charge of the electrophoretic colorants or bounding capsule. Reactants or solvents for the microencapsulation process (oil soluble monomers, for example) can also be contained in the suspending fluid. Charge control agents can also be added to the suspending fluid.

The suspension medium must be capable of being formed into small droplets prior to a capsule being formed. Processes for forming small droplets include flow-through jets, membranes, nozzles, or orifices, as well as shear-based emulsifying schemes. The formation of small drops may be assisted by electrical or sonic fields. Surfactants and polymers can be used to aid in the stabilization and emulsification of the droplets in the case of an emulsion type encapsulation.

As noted above, it may be advantageous for the suspension medium to contain an optically absorbing dye. This dye must be soluble in the suspension medium, but will generally be insoluble in the other components of the capsule. There is much flexibility in the choice of dye material provided that the dyes are cosmetically suitable and do not interfere with the electrophoretic colorants.

Encapsulation

Following the incorporation of the electrophoretic colorants within the suspension medium, the suspension may be encapsulated in those embodiments where a colorant capsule is desired. Encapsulation of the electrophoretic colorant and suspension medium may be accomplished by one of numerous suitable encapsulation procedures known in the art and detailed within Microencapsulation, Processes and Applications, (I. E. Vandegaer, ed.), Plenum Press, New York, N.Y. (1974); Gutcho, Microcapsules and Mircroencapsulation Techniques, Nuyes Data Corp., Park Ridge, N.J. (1976), J. Colloid and Int. Science, V44, N1, pp. 133, July 1973, U.S. Pat. Nos. 2,800,457; 4,001,140; 4,087,376; 4,273,672; and 5,320,835; and U.S. Patent Publication No. US 2002/0180687 A1, all of which are hereby incorporated by reference herein. The processes fall into several general categories, all of which can be applied to the present invention: interfacial polymerization, in situ polymerization, physical processes, such as coextrusion and other phase separation processes, in-liquid curing, and simple/complex coacervation.

Numerous materials and processes should prove useful in formulating the colorant microcapsules for the cosmetic composition of the present invention. Useful materials for simple coacervation processes include, but are not limited to, gelatin, polyvinyl alcohol, polyvinyl acetate, glutaraldehyde, calcium alginate, polymers (such as EMULSAN™) and cellulosic derivatives, such as, for example, carboxymethylcellulose. Useful materials for complex coacervation processes include, but are not limited to, gelatin, acacia, carageenan, carboxymethylcellulose, hydrolized styrene anhydride copolymers, agar, alginate, casein, albumin, methyl vinyl ether co-maleic anhydride, and cellulose phthalate. Useful materials for phase separation processes include, but are not limited to, polystyrene, PMMA, polyethyl methacrylate, polybutyl methacrylate, ethyl cellulose, polyvinyl pyridine, and poly acrylonitrile. Useful materials for in situ polymerization processes include, but are not limited to, polyhydroxyamides, with aldehydes, melamine, or urea and formaldehyde; water-soluble oligomers of the condensate of melamine, or urea and formaldehyde; and vinyl monomers, such as, for example, styrene, MMA and acrylonitrile. Finally, useful materials for interfacial polymerization processes include, but are not limited to, diacyl chlorides, such as, for example, sebacoyl, adipoyl, and di- or poly-amines or alcohols, and isocyanates. Useful emulsion polymerization materials may include, but are not limited to, styrene, vinyl acetate, acrylic acid, butyl acrylate, t-butyl acrylate, methyl methacrylate, and butyl methacrylate.

In the context of the present invention, one skilled in the art will select an encapsulation procedure and wall material based on the desired capsule properties. These properties include the distribution of capsule radii; electrical, mechanical, diffusion, and optical properties of the capsule wall; and chemical compatibility with the suspension medium of the capsule.

The microcapsules of the current invention should be hollow and may have a cubical, cylindrical or spherical shape. The diameter of the microcapsules should be less than about 200 μm in diameter, preferably less than about 100 μm, and most preferably less than about 50 μm in diameter. The microcapsule wall should also be mechanically strong (although if the finished capsule powder is to be dispersed in a curable polymeric binder for coating, mechanical strength is not as critical). The microcapsule wall should generally not be porous. If however, it is desired to use an encapsulation procedure that produces porous microcapsules, these can be overcoated in a post-processing step (i.e., a second encapsulation). Moreover, if the microcapsules are to be dispersed in a curable binder, the binder will serve to close the pores. The microcapsule walls should be optically clear. The wall material may, however, be chosen to match the refractive index of the suspension medium of the capsule (i.e., the suspending fluid) or a cosmetic vehicle in which the microcapsules are to be dispersed.

Within the capsule the electrophoretic colorants should comprise about 5-60%, preferably about 10-50%, and most preferably about 15-40% by weight of the microcapsule.

Cosmetic Vehicle

The colorant microcapsules or electrophoretic colorants, depending on whether the encapsulated or unencapsulated embodiment of the cosmetic is being manufactured, may be suspended within a cosmetically acceptable vehicle. Such vehicles may take the form of any known in the art suitable for application to skin including lips, nails, or hair including hair of the scalp, facial hair, eyelashes, and eyebrows, and may include water (e.g., deionized water); vegetable oils; mineral oils; esters such as octal palmitate, isopropyl myristate and isopropyl palmitate; ethers such as dicapryl ether and dimethyl isosorbide; isoparaffins such as isooctane, isododecane and isohexadecane; silicone oils such as cyclomethicone, dimethicone, dimethicone cross-polymer, polysiloxanes, and their derivatives, preferably organomodified derivatives; hydrocarbon oils such as mineral oil, petrolatum, isoeicosane, and polyisobutene; polyols such as propylene glycol, glycerin, butylene glycol, pentylene glycol, and hexylene glycol; waxes such as beeswax and botanical waxes; or any combinations or mixtures of the foregoing.

The vehicle may comprise an aqueous, polyol or hydropolyol phase, an oil phase, a silicone phase, and suitable combinations thereof. The cosmetically acceptable vehicle may comprise an aqueous, polyol, or hydropolyol gel composition, or the cosmetically acceptable vehicle may also comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like, for example, having the appearance of a cream, gel or micro-emulsions. The emulsion may include an emulsifier, such as a nonionic, anionic or amphoteric surfactant. Oil-in-water emulsions are preferred.

The aqueous phase of the emulsion may include water, one or more additional water soluble solvents such as polyols, and one or more water soluble or water dispersible active components. The aqueous phase of the emulsion also typically contains the colorant microcapsule and/or electrophoretic colorant, which are suspended or dispersed therein. The cosmetically acceptable vehicle can comprise component(s) compatible with the system used. For example, polyols, preferably propylene glycol, can form a polymer suspension or dispersion as hereinbefore described, in combination with or without water, which suspension/dispersion is subsequently incorporated into the cosmetic composition.

In some embodiments, the cosmetic formulation may contain an oil phase, wax, and/or an emulsion. Formulations corresponding to other types of cosmetics, for example, foundations or lip products, may include an oil phase and/or an emulsion. In further embodiments, the formulation does not comprise an oil or an oil phase. In some embodiments, the formulation does not comprise an emulsion.

The water phase of the emulsion preferably has one or more organic compounds, including emollients; humectants (such as butylene glycol, propylene glycol, Methyl gluceth-20, and glycerin); other water-dispersible or water-soluble components including thickeners such as Veegum or hydroxyalkyl cellulose; gelling agents, such as high MW polyacrylic acid, i.e. CARBOPOL 934; and mixtures thereof. The emulsion may have one or more emulsifiers capable of emulsifying the various components present in the composition.

Compounds suitable for use in the oil phase include without limitation, vegetable oils; esters such as octyl palmitate, isopropyl myristate and isopropyl palmitate; ethers such as dicapryl ether; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; natural or synthetic waxes; one or more oil soluble active components, and the like, individually or in compatible combination. Suitable hydrophobic hydrocarbon oils may be saturated or unsaturated, have an aliphatic character and be straight or branched chained or contain alicyclic or aromatic rings. The oil-containing phase may be composed of a singular oil or mixtures of different oils.

Hydrocarbon oils include those having 6-20 carbon atoms, more preferably 10-16 carbon atoms. Representative hydrocarbons include decane, dodecane, tetradecane, tridecane, and C8-20 isoparaffins. Paraffinic hydrocarbons are available from Exxon under the ISOPARS trademark, and from the Permethyl Corporation. In addition, C8-20 paraffinic hydrocarbons such as C12 isoparaffin (isododecane) manufactured by the Permethyl Corporation having the tradename Permethyl 99ATM are also contemplated to be suitable. Various commercially available C16 isoparaffins, such as isohexadecane (having the tradename Permethyl®) are also suitable. Examples of preferred volatile hydrocarbons include polydecanes such as isododecane and isodecane, including for example, Permethyl-99A (Presperse Inc.) and the C7-C8 through C12-C15 isoparaffins such as the Isopar Series available from Exxon Chemicals. A representative hydrocarbon solvent is isododecane.

The oil phase may comprise one or more waxes, including for example, rice bran wax, carnauba wax, ouricurry wax, candelilla wax, montan waxes, sugar cane waxes, ozokerite, shellac wax, rice bran wax, polyethylene waxes, Fischer-Tropsch waxes, beeswax, botanical waxes, microcrystalline wax, silicone waxes, fluorinated waxes, paraffin wax, synthetic waxes, and any combination thereof “Wax” or “waxes”, as used herein, generally refers to compounds that are solid at room temperature (about 25° C.), and having a melting point ranging from about 45° C. to about 110° C. The wax component may be incorporated into the compositions of the invention in an amount of up to about 25% by weight, typically from 0 to about 20 weight %, from about 0.5 to about 15 weight %, and from about 1 to about 12 weight %. Suitably, the compositions can contain 2, 4, 6, 8, 10, or 12 weight % wax. For example, in some particularly preferred embodiments, a cosmetic composition is provided that comprises from about 2 to about 12 weight % of waxes and about 4 to about 6 weight % of the polymer of Rheolate® 288 in a water/propylene glycol aqueous system, where the polymer/aqueous system itself comprises about 20 weight % polymer; about 40 weight % water; and about 40 weight % propylene glycol. In some other particularly preferred embodiments, a cosmetic composition is provided that comprises from about 0 to about 24% waxes and from about 2 to about 10 weight % of the polymer of Rheolate® 288 in a water/propylene glycol aqueous system, where the polymer/aqueous system itself comprises about 20 weight % polymer; about 40 weight % water; and about 40 weight % propylene glycol. In some even more preferred embodiments, a cosmetic composition is provided that comprises from about 1.5 to about 12 weight % waxes and about 5 weight % of the polymer of Rheolate® 288 in a water/propylene glycol aqueous system, where the polymer/aqueous system itself comprises about 20 weight % polymer; about 40 weight % water; and about 40 weight % propylene glycol. See Example 3 below. In some other even more preferred embodiments, a cosmetic composition is provided that comprises about 12 weight % wax and about 2.5 weight % of the polymer of Rheolate® 288 in a water/propylene glycol aqueous system, where the polymer/aqueous system itself comprises about 30 weight % polymer; about 15 weight % water; and about 55 weight % propylene glycol.

The oil phase may comprise one or more volatile and/or non-volatile silicone oils. Volatile silicones include cyclic and linear volatile dimethylsiloxane silicones. In some embodiments, the volatile silicones may include cyclodimethicones, including tetramer (D4), pentamer (D5), and hexamer (D6) cyclomethicones, or mixtures thereof. Particular mention may be made of the volatile cyclomethicone-hexamethyl cyclotrisiloxane, octamethyl-cyclotetrasitoxane, and decamethyl-cyclopentasitoxane. Suitable dimethicones are available from Dow Corning under the name Dow Corning 200® Fluid and have viscosities ranging from 0.65 to 600,000 centistokes or higher. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics”, Cosmetics and Toiletries, 91:27-32 (1976), herein incorporated by reference in its entirety. Linear volatile silicones generally have a viscosity of less than about 5 centistokes at 25° C., whereas the cyclic silicones have viscosities of less than about 10 centistokes 25° C. Examples of volatile silicones of varying viscosities include Dow Corning 200, Dow Corning 244, Dow Corning 245, Dow Corning 344, and Dow Corning 345, (Dow Corning Corp.); SF-1204 and SF-1202 Silicone Fluids (G.E. Silicones), GE 7207 and 7158 (General Electric Co.); and SWS-03314 (SWS Silicones Corp.). Linear, volatile silicones include tow molecular weight polydimethylsilaxane compounds such as hexamethyldisiloxane, octamethyltrisilaxane, decamethyltetrasiloxane, and dodecamethylpentasiloxane, to name a few.

Non-volatile silicone oils will typically comprise polyalkylsiloxanes, polyarylsiloxanes, polyalkylarylsiloxanes, or mixtures thereof. Polydimethylsiloxanes are preferred non-volatile silicone oils. The non-volatile silicone oils will typically have a viscosity from about 10 to about 60,000 centistokes 25° C., preferably between about 10 and about 10,000 centistokes, and more preferred still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted will various functional groups such as alkyl, aryl, amine groups, vinyl, hydroxyl, haloalkyl groups, alkylaryl groups, and acrylate groups, to name a few. Based on the teachings herein, a person skilled in the art will be able to select any of these silicone oils or other optional additives, and/or the amount thereof, such that the desirable properties of the cosmetic compositions described herein can be conserved.

Non-limiting emulsifiers include emulsifying waxes, polyether polyols, polyethers, mono- or di-ester of polyols, ethylene glycol mono-stearates, glycerin mono-stearates, glycerin di-stearates, silicone-containing emulsifiers, soya sterols, acrylate, fatty acids such as stearic acid, fatty acid salts, and mixtures thereof. The preferred emulsifiers include soya sterol, stearic acid, emulsifying wax, acrylates, silicone containing emulsifiers and mixtures thereof. Other specific emulsifiers that can be used in the composition of the present invention include, but are not limited to, one Or more of the following: C10-30 alkyl acrylate crosspolymer; Dimethicone PEG-7 isostearate; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2. Steareth-10, and Steareth-20, to name a few. Additional emulsifiers are provided in the INCI Ingredient Dictionary and Handbook 11th Edition (2006), the disclosure of which is hereby incorporated by reference.

These emulsifiers typically will be present in the composition in an amount from about 0.001% to about 10% by weight, in particular in an amount from about 0.01% to about 5% by weight, and more preferably, from about 0.1% to about 3% by weight.

The water-in-silicone emulsion may be emulsified with a nonionic surfactant (emulsifier) such as, for example, polydiorganosiloxane-polyoxyalkylene block copolymers, including those described in U.S. Pat. No. 4,122,029, the disclosure of which is hereby incorporated by reference. These emulsifiers generally comprise a polydiorganosiloxane backbone, typically polydimethylsiloxane, having side chains comprising —(EO)m- and/or —(PO)n- groups, where EO is ethyleneoxy and PO is 1,2-propyleneoxy, the side chains being typically capped or terminated with hydrogen or lower alkyl groups (e.g., C1-6, typically C1-3). Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABIL EM 90 and EM 97, available from Goldschmidt Chemical Corporation (Hopewell, Va.); and the SILWET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasitoxane. Preferred examples of water-in-silicone emulsifiers include, without limitation. PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name Abil EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The water-in-silicone emulsifiers typically will be present in the composition in an amount from about 0.001% to about 10% by weight, in particular in an amount from about 0.01% to about 5% by weight, and more preferably, below 1% by weight. A person of skill in the art, based on the teachings herein, will be able to select any of these emulsifiers or other optional additives, and/or the amount thereof, such that the desirable properties of the cosmetic compositions described herein can be conserved.

The oil-containing phase of emulsions useful herein will typically comprise from about 1% to about 75%, preferably from about 5% to about 50%, and more preferably from about 20% to about 25% by weight, based on the total weight of the emulsion; and the aqueous phase will typically comprise from about 25% to about 99%, preferably from about 50% to about 95%, and more preferably from about 75% to about 80% by weight of the total emulsion. The aqueous phase will typically comprise from about 25% to about 100%, more typically from about 50% to about 95%, or often from about 40% to about 80% by weight water by weight water.

The composition of various embodiments of the invention may optionally comprise other cosmetic actives and excipients, obvious to those skilled in the art including, but not limited to, masking agents, medicaments, moisturizers, pH adjusters, protectants, soothing agents, viscosifiers, fillers, emulsifying agents, antioxidants, surfactants, chelating agents, gelling agents, thickeners, emollients, humectants, moisturizers, vitamins, minerals, viscosity and/or additional rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfoilients, lubricants, fragrances, colorants, depigmenting agents, hypopigmenting agents, preservatives (e.g., DMDM Hydantoin/Iodopropynylbutylcarbonate), stabilizers, pharmaceutical agents, photostabilizing agents, neutralizers (e.g., triethanolamine) and mixtures thereof.

Thickeners may include, for example, cellulose-based thickeners, for example, water-soluble cellulose-based thickeners, such as hydroxyethylcellulose, methylcellulose, hydroxypropylcellulose and carboxymethylcellulose; gums, for example, gums sold under the name “Cellosize QP 4400 H” by the company Amerchol; guar gum, for example, those sold under the name Vidogum GH 175 by the company Unipectine and under the name Jaguar C by the company Meyhall; quaternized guar gum sold under the name “Jaguar C-13-S” by the company nonionic guar gums comprising C1-C6 hydroxyalkyl groups, such as, for example, hydroxymethyl, hydroxyethyl, hydroxypropyl and hydroxybutyl groups, like the guar gums sold under the trade names Jaguar HP8, Jaguar HP60, Jaguar HP120, and Jaguar HP 105 by the company Meyhall or under the name Galactasol 40H4FD2 by the company Aqualon; xanthan gum, carob gum, scleroglucan gum, gellan gum, rhamsan gum, and karaya gum; alginates, maltodextrin, starch and its derivatives, hyaluronic acid and its salts; clays, for example, montmorillonites, hectorites, and laponites; crosslinked polyacrylic acids, such as the “Carbopol” products from the company Goodrich; the polyglyceryl(meth)acrylate polymers sold under the names “Hispagel” or “Lubragel” by the companies Hispano Quimica or Guardian; polyvinylpyrrolidone; crosslinked acrylamide polymers and copolymers, such as those sold under the names “PAS 5161” or “Bozepol C” by the company Hoechst, or “Sepigel 305” by the company SEPPIC; crosslinked methacryloyloxyethyltrimethylammonium chloride homopolymers sold under the name “Salcare SC95” by the company Allied Colloid; and the like. Based on the teachings herein, a person skilled in the art will be able to select any of these or other optional additives, and/or the amount thereof, such that the desirable properties of the cosmetic compositions described herein are conserved.

The composition can also comprise other ingredients usually used in cosmetics. Such ingredients can be chosen, in particular, from plasticizers, coalescence agents, fillers, dyestuffs, such as pigments or dyes, surfactants, preserving agents, oils, cosmetic agents, such as moisturizers and anti-UV agents that are well known in the art.

Various fillers and additional components may be added. Fillers are normally present in an amount from about 0 weight % to about 20 weight %, based on the total weight of the composition, preferably from about 0.1 weight % to about 10 weight %. Suitable fillers include without limitation silica, treated silica, talc, zinc stearate, mica, kaolin, Nylon powders such as Orgasol™, polyethylene powder, Teflon starch such as rich starch, boron nitride, copolymer microspheres such as Expancel™ (Nobel Industries), Polytrap™ (Dow Corning) and silicone resin microbeads (Tospearl™ from Toshiba), polytetrafluoroethylene, and the like. Fillers may be selected to be are compatible with an aqueous medium, where the composition is provided in such, including, in particular the fillers starch, talc and polytetrafluoroethylene. Cosmetic compositions that include an oil phase, e.g., a wax, can use other fillers suitable for non-aqueous systems.

Electric Field

As noted above, the color-changeable cosmetic composition of the current invention is responsive to electric fields—the electrophoretic colorants move relative to an electric field applied to the cosmetic. Preferably, the electric field is applied by a device similar in design to common cosmetic applicators including but not limited to wands, brushes, sponges, pens, markers, etc. Each of these applicators have a means of holding the applicator such as a handle, strap, shaft, etc., and a head, tip, point etc. at which the electric field is applied to the color-changeable cosmetic. The head of the applicator is preferably shaped to accommodate its intended use, i.e., an applicator intended to act as a lip or eye liner would have a fine point, whereas an applicator for foundation or eye shadow would have a larger head to effect the change over a larger area, and the applicator may be in the form of a comb or brush for effecting the change when the color changeable composition is used as a hair colorant.

The electric field/charge for the applicator may be provided by any electrical means known in the art including piezoelectric, electrochemical, thermoelectric, photoelectric, and/or triboelectric charging (static electricity). In preferred embodiments the electric field may be generated by triboelectric charging. Triboelectric charging is a contact electrification that occurs to a material when it comes into contact (such as by rubbing) with a different material and the materials become electrically charged. The polarity and strength of the charges produced differ according to the materials, surface roughness, temperature, strain, and other properties.

For example, the following materials can be used depending upon the desired charge:

TABLE 1
Tribioelectric materials

| | |
|---|---|
| Most positive | Leather |
| | Rabbit's fur |
| | Glass |
| | Quartz |
| | Mica |
| | Human hair |
| | Nylon |
| | Wool |
| | Lead |
| | Cat's fur |
| | Silk |
| | Aluminium |
| | Paper |
| | Cotton |
| Zero | Steel |
| | Wood |
| | Lucite |
| | Amber |
| | Sealing wax |
| | Acrylic |
| | Polystyrene |
| | Rubber balloon |
| | Hard rubber |
| | Nickel, Copper |
| | Sulfur |
| | Brass, Silver |
| | Acetate, Rayon |
| | Synthetic rubber |
| | Polyester |
| | Styrene (Styrofoam) |
| | Orlon |
| | Plastic wrap |
| | Polyurethane |
| | Polyethylene (like Scotch tape) |
| | Polypropylene |
| | Vinyl (PVC) |
| | Silicon |
| | Teflon |
| | Silicone rubber |
| Most negative | Ebonite |

If two items from the list are rubbed together, then the item that is higher on the list will end up more positively charged and the lower one will end up more negatively charged. Thus, in order to generate a positive charge, the applicators head can be made of glass and rubbed against silicone rubber or plastic wrap; and conversely to generate a negatively charged applicator the head could be made of Teflon and rubbed against nylon or Rabbits Fur.

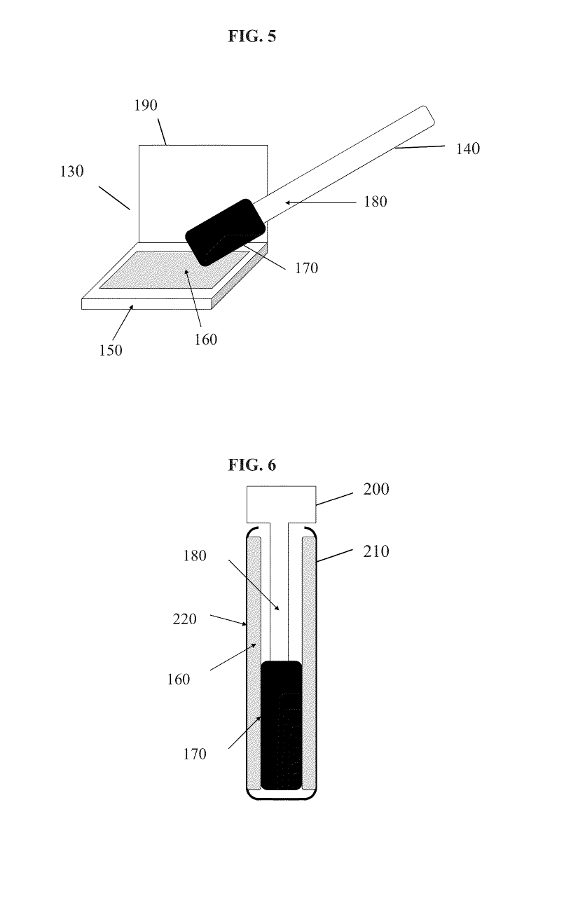

A further embodiment of the current invention is directed to a triboelectric applicator particularly suited for use with the inventive color-changeable cosmetic composition. FIGS. 5 and 6 illustrate two similar embodiments of triboelectric applicators. FIG. 5 provides an embodiment similar to a cosmetic compact having a container 130 having a cover 190 and base 150. The base contains a mating surface 160 upon which the head of an applicator 170 can be rubbed to generate the desired charge. The user would grasp the applicator 140 by a handle 180 and rub the head of the applicator 170 against the mating surface 160 to generate an electric charge before applying that charge to an area of the wearer's skin coated by the color-changeable cosmetic. FIG. 6 illustrates an electric field applicator in the shape of a mascara tube. The mating surface 160 in this embodiment lines the interior of a tube/cylinder 220 such that when the applicator's head 170 is rubbed against the mating surface 160 it adopts a charge.

Further embodiments of the electric Field applicators are contemplated that resemble other common cosmetic applicators such as a lip stick tube where the mating surface would line the tube and the head would be on the extendible cylinder such that when the cylinder was extended in a fashion similar to lip stick the head would be charged. Similarly, an eye liner pen could be adopted such that the interior of the cap for the eyeliner pen would be covered by a mating surface and the head of the eye liner could be charged when removing the cap. In further embodiments where accessories, i.e. acrylic/press-on nails, false eyelashes, hair extensions, etc. incorporate the electrophoretic colorants of the current invention the accessory may include an electric means such as a piezoelectric generator to permit color changes to occur when the wearer desires by pressing on the accessory itself or permit color changes to occur at regular intervals independent of the wearers actions. Additionally, the applicators may be modified to have two or more mating and head surfaces such that the wearer would be able to generate different charges (+/−) and different intensities of charges for purposes of effecting as many color changes as the color changeable colorant will permit. Further, the mating surface may be replaced with a Van de Graaf generator within the applicator which can generate the necessary charge.

In a further application of the invention, the containers for the color changeable cosmetics of the current invention may incorporate with suitable triboelectric, piezoelectric, etc. means to effect changes in the color of the cosmetic for display purposes to illustrate to the consumer the various color options offered by the cosmetic.

Additionally, a further embodiment of the current invention is directed to a cosmetic kit in which the electric field applicator and the color-changeable cosmetic are provided within a single package such as a compact for an eyeliner.

Cosmetic Formulations Using Color-Changeable Cosmetics

The compositions according to the instant invention can be formulated in a variety of forms for topical application. The composition may be formulated in a variety of product forms suitable for application to the skin, hair, eyelashes, or eyebrows, such as, for example, a lotion, cream, serum, spray, aerosol, ointment, essence, gel, paste, patch, pomade, solution, towelette, mask, foam, elixir, concentrate, or any other liquid or semisolid form.

Suitable forms may depend on the type of cosmetic product. For example, for a foundation, the composition is preferably formulated as a lotion, cream, liquid, or mousse; for an eye eyeliner, the composition is preferably formulated as a liquid; for an eye shadow, the composition is preferably formulated as a cream; for a lip product, the composition is preferably formulated as a paste or cream; for a mascara product, the composition is preferably formulated as a paste or cream, preferably supplied in a reservoir with an applicator, wand, or brush integral with a removable closure.

In one embodiment of the current invention, a pink lipstick incorporating the color changeable cosmetic of the current invention is applied to a wearer's lips. The wearer may subsequently utilize the electric field applicator to adjust the color intensity or shade of the lipstick. For example, adjusting the pink color to more of a reddish shade. Alternatively, the wearer may change the color of the lipstick altogether such that user may apply the make-up once and be able to adjust the color to suit the occasion, for example adjusting the color of the lipstick from a more formal color (dark red) to that of a more casual color (pink/peach) to attend after work social events.

In further embodiments, the electrophoretic colorants may be incorporated into a tattoo ink, nail polish, acrylic nails, etc. The user through the application of an electric field may be able to adjust the color and/or pattern present on these adornments to suit their fancy over the lifetime of these adornments.

In yet a further embodiment of the current invention, the color-changeable cosmetics of the current invention may be used as part of a method of detecting and correcting the appearance of damaged skin. An example of the method is demonstrated within FIGS. 7A and 7B. In FIG. 7A a color-changeable cosmetic (having red (−) electrophoretic colorants 230 and black (+) electrophoretic colorants 240) of the current invention is applied to healthy skin Healthy skin normally exhibits a negative electric field and thus the red (−) electrophoretic colorants 230 are repelled from the surface contacting the skin and are displayed on the display surface of the cosmetic. However, as FIG. 7B demonstrates, when skin is damaged or injured the damaged skin loses this negative electric charge. When this occurs, the red (−) electrophoretic colorants 230 are drawn to the contact surface and the black (+) electrophoretic colorants 240 are repelled to the display surface, as shown in FIG. 7B. The area of damaged skin is then identified by the black patches on the otherwise red field. In alternative embodiment, a color-changeable cosmetic of FIG. 2 is utilized. A non-limiting list of skin damage identifiable by this method include chronologically aged skin, photo-aged skin, hormonally aged, and/or actinic aged skin, atrophied skin, areas with impaired microcirculation, cracked skin or areas where the skin barrier has been impaired, bruised, fatigued and/or stressed skin; and environmentally stressed skin. In further embodiments, the color-changeable cosmetic may be used diagnostically to identify disease areas of the skin suffering from or at risk of developing afflictions or maladies including, but not limited to, skin cancer, skin lesions, acne, psoriasis, warts, etc. Further, this sensitivity to the electric charge of the skin permits color-changeable cosmetic formulations to be made which will cover solely the damaged areas of the skin since the color change is initiated by the electric field of damaged skin thus permitting the damaged areas to be covered-up while still presenting a natural look. Furthermore, in the normal use of the cosmetic compositions of the invention, the skin will not be universally consistent over the wearer's body, and thus the coloring provided by the color-changeable cosmetic of the current invention may not be uniform and present a pixilated look that is more natural.

The illustrative examples set forth herein further describe and demonstrate illustrative embodiments within the scope of the present invention. The examples are given solely for the purpose of illustration and are not to be constructed as limitations of the present invention, as many variations thereof are possible without departing from the spirit and scope of the invention.

### Example 1

### Preparation of the Colorant Capsule

TABLE 2
Materials for colorant microcapsule.

| | Ingredient | Weight % |
|---|---|---|
| Particles: | Polymer coated TiO2 | 30 |
| | Polymer coated Black Iron Oxide | 10 |
| Suspension medium: | Low viscosity oil | 60 |
| | Total | 100 |

The particles may then be dispersed in the suspension medium using high shear such as milling, sonication or three roll mill.

The above dispersion may then be microencapsulated using a standard coacervation technique (for examples, see U.S. Pat. No. 4,752,496, U.S. Pat. No. 5,320,835, and Journal of Colloid and Interface Scien, Vol 44, No 1, July 1973, pp 133-141). The microcapsules (electrophoretic particles) were formulated as follows:

TABLE 3
Coacervation Formula

| Ingredient | Weight Percent |
|---|---|
| Particle dispersion from (a) | 84.9 |
| Gelatin | 3.5 |
| Gum Arabic | 4.0 |
| Urea formaldehyde polymer | 6.0 |
| Glutaraldehyde | 0.1 |
| Silica | 1.5 |
| Total | 100 |

The microcapsules may be obtained in the form of stable, dry, free flowing powder.

### Example 2

### Preparation of a Lipstick Incorporating the Colorant Capsules from Example 1

The particles from Example 1 in a lipstick formula in the proportions described below:

TABLE 4
Lipstick Formulation

| Ingredient | Weight Percent |
|---|---|
| Cosmetic Vehicle | |
| Isododecane | 27% |
| Acrylate copolymer | 12% |
| Ozokerite | 4% |
| Polyethylene Wax | 7% |
| Diisotearyl Fumarate | 21% |
| Red iron oxide* | 5% |
| D&C Lake* | 2% |
| Octyldodecanol | 5% |
| Stearyl Dimethicone | 5% |
| Ethylhyxylmethocinnamate | 7% |
| Total | 95% |
| Electrophoretic Colorant | |
| Colorant microcapsules | 5% |
| Total | 100% |

*Non-electrophoretic

All the ingredients, except the colorant microcapsules, may be mixed on an overhead stirrer at 80° C. until homogenous. The pigments—Red iron oxide and D&C lake—may be predispersed in stearyl dimethicone or another dispersing solvent using a three roll mill or another technique well known in the art. Thereafter, the colorant microcapsules may be added to the formulation with overhead stirring and the mixture is allowed to come to room temperature while sweeping.

The above cosmetic can then be laid down on a keratinous substrate such as hair or skin. Upon application of the wand described in Case B, the negative charge of the wand will attract the positively charged (TiO₂, white in color) to the surface of the microcapsule and result in a brighter (lighter) color development on the keratinous substrate.

### Example 3

### Preparation of a Foundation Incorporating the Colorant Capsules from Example 1

The particles from Example 1 in a foundation formula in the proportions described below:

TABLE 5
Foundation Formulation

| Phase | Ingredient | Weight Percent |
|---|---|---|
| Phase A | Demineralized Water | 55.0% |
| | Xantham gum | 0.50% |
| | Veegum | 0.50% |
| | Butylene Glycol | 5.71% |
| | Methylparaben | 0.39% |
| | Sodium Hexametaphosphate | 0.24% |
| | Tetrasodium EDTA | 0.10% |
| Phase B | Iron Oxide-Yellow | 0.45% |
| | Iron Oxide-Black | 0.09% |
| | Iron Oxide-Red | 0.15% |
| | Titanium Dioxide | 6.67% |
| Phase C | Sorbitan Monostearate | 2.42% |
| | Propylene Glycol Dicaprylate | 4.84% |
| | Ethylene Glycol Monostearate | 3.15% |
| | Myristal Ether Propionate | 2.42% |
| | Sorbitan Monostearate | 1.21% |
| | Cyclomethicone | 5.78% |
| Phase D | Imidazolidinyl Urea | 0.39% |
| Phase E | Colorant Microcapsule | 10.00% |
| | Total | 100.00% |

Phase A and C may be mixed using an overhead stirrer separately at 80° C. Upon obtaining homogenous mixture, phase B may be added to phase A and milled for 10 minutes. Thereafter, phase C can be added while milling and the mixture is allowed to emulsify. Phase B and E can then be added using an overhead stirrer and the mixture is allowed to cool to 50° C. and phase D can then be added. The mixture is allowed to cool to room temperature using an overhead stirrer.

The above cosmetic can be laid down on a keratinous substrate such as skin or hair. Upon application of the wand described in Case A, the positive charge of the wand will attract the negatively charged (Black iron oxide, black in color) to the surface of the microcapsule and result in a deeper (darker) color development of the cosmetic film. Alternatively, the wand described in Case B can be used to develop a lighter color. This gives the flexibility to obtain on demand shade shift for the entire or part of the cosmetic film.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fail within the scope of the appended claims. All publications cited herein are incorporated by reference in their entirety.

1. A stable and reversible color-changeable cosmetic composition for application to a human integument having a first color when applied comprised of at least one electrophoretic colorant having a color, a charge and a zeta potential within a suspension medium, wherein when a first electric field source having a charge is placed in proximity to the color-changeable cosmetic the electrophoretic colorant moves relative to the first electric field source within a desired time to effect a change in the color-changeable cosmetic to a second color and wherein upon application of a second electric field source the color of the cosmetic composition may be further modified.

2. The color-changeable cosmetic composition of claim 1, wherein the suspension medium is a suitable cosmetic vehicle.

3. The color-changeable cosmetic composition of claim 2, wherein the suitable cosmetic composition is an emulsion.

4. The color-changeable cosmetic composition of claim 2, wherein the suitable cosmetic vehicle has an initial viscosity of less than about 100,000 centipoise.

5. The color-changeable cosmetic composition of claim 1, wherein the suspension medium has an opaque color.

6. The color-changeable cosmetic composition of claim 5, wherein the first color is the opaque color of the suspension medium, and the second color is the color of the electrophoretic colorant.

7. The color-changeable cosmetic composition of claim 1, further comprised of more than one electrophoretic colorant wherein each electrophoretic colorant has a color, a charge, and a zeta potential.

8. The color-changeable cosmetic composition of claim 7, further comprised of two electrophoretic colorants, a first electophoretic colorant having a first electrophoretic colorant color, a first electrophoretic charge, and a first zeta potential and a second electrophoretic colorant having a second electrophoretic colorant color, a second electrophoretic colorant charge, and a second zeta potential.

8. The color-changeable cosmetic composition of claim 7, wherein the first electrophoretic colorant charge is different than the second electrophoretic colorant charge.

9. The color-changeable cosmetic composition of claim 1, wherein the at least one electrophoretic colorant has a zeta potential greater than about 10 mV.

10. The color-changeable cosmetic of claim 7, wherein the more than one electrophoretic colorants each have a different color.

11. The color-changeable cosmetic composition of claim 7, wherein the more than one electrophoretic colorants have the same charge and non-overlapping zeta potentials.

12. The color-changeable cosmetic composition of claim 11, wherein the non-overlapping zeta potentials are separated by at least about 2 mV.

13. The color-changeable cosmetic composition of claim 1, wherein the electric field source is the human integument.

14. The color-changeable cosmetic composition of claim 13, wherein the electric field source is skin.

15. The color-changeable cosmetic composition of claim 1, wherein the at least one electrophoretic colorant and the suspending medium are contained within a microcapsule.

16. The color-changeable cosmetic composition of claim 15, wherein the microcapsule is cubical, cylindrical, or spherical.

17. The color-changeable cosmetic composition of claim 15, wherein the diameter of the microcapsule is less than about 100 μm.

18. The color-changeable cosmetic composition of claim 1, wherein the desired time is between about 0.01 to 5 seconds.

19. A method for providing a color changeable cosmetic to an integument wherein:
(i) the color-changeable cosmetic of claim 1 having a first color is applied to an integument; and
(ii) a first electric field source is applied to the cosmetic composition to effect a change in color of the cosmetic composition to a second color and wherein upon the application of a second electric field source the color of the cosmetic composition may be further modified.

20. The method of claim 19, wherein the first electric field source is the skin.

21. The method of claim 20, wherein the change in the cosmetic composition identifies a damaged or injured portion of the integument.

22. The method of claim 19, wherein the damaged or injured integument is selected from the group consisting of chronologically aged skin, photo-aged skin, hormonally aged, and/or actinic aged skin, atrophied skin, areas with impaired microcirculation, cracked skin or areas where the skin barrier has been impaired, bruised, fatigued and/or stressed skin; and environmentally stressed skin.

23. The method of claim 22, wherein the damaged or injured integument is skin suffering from or at risk of developing an affliction or a malady.

24. The method of claim 23, wherein the affliction or malady is selected from the group consisting of skin cancer, skin lesions, acne, psoriasis, or warts.

25. A kit for a color changeable cosmetic comprised of:
a. a cosmetic composition of claim 1; and
b. a triboelectric field source applicator comprised of:
i. a case having a mating surface therein;
ii. a wand having a head surface thereon;
wherein the mating surface and head surface are made of materials possessing different charges and when the head surface is rubbed against the mating surface a charge is generated.

26. The kit of claim 25, wherein the triboelectric field source applicator has a teflon head surface.

27. The kit of claim 26, wherein the triboelectric field source applicator has a nylon or rabbits fur mating surface.

28. The kit of claim 25, wherein the triboelectric field source applicator has a glass head surface.

29. The kit of claim 28, herein the triboelectric field source applicator has a silicone rubber mating surface.
